SEQUENCE LISTING (35 sequences)

SEQ ID NO: 1 - DNA, 16 bp, Artificial Sequence
  Synthetic oligonucleotide
cgcgtcggaaggggtc

SEQ ID NO: 2 - DNA, 18 bp, Artificial Sequence
  Synthetic oligonucleotide
actcgttcccgacgctcg

SEQ ID NO: 3 - DNA, 28 bp, Artificial Sequence
  Synthetic oligonucleotide
cgttttataggttaagtgcgttgtgttc

SEQ ID NO: 4 - DNA, 22 bp, Artificial Sequence
  Synthetic oligonucleotide
attgcgctatctcgtccgaacg

SEQ ID NO: 5 - DNA, 21 bp, Artificial Sequence
  Synthetic oligonucleotide
gttcgggatttcgatttggac

SEQ ID NO: 6 - DNA, 16 bp, Artificial Sequence
  Synthetic oligonucleotide
ccaccgaatcgcgcag

SEQ ID NO: 7 - DNA, 22 bp, Artificial Sequence
  Synthetic oligonucleotide
tggaattttcggttgattggtt

SEQ ID NO: 8 - DNA, 19 bp, Artificial Sequence
  Synthetic oligonucleotide
aacaacgtccgcacctcct

SEQ ID NO: 9 - DNA, 21 bp, Artificial Sequence
  Synthetic oligonucleotide
gcgtcggaggttaaggttgtt

SEQ ID NO: 10 - DNA, 22 bp, Artificial Sequence
  Synthetic oligonucleotide
ctctccaaaattaccgtacgcg

SEQ ID NO: 11 - DNA, 21 bp, Artificial Sequence
  Synthetic oligonucleotide
gggttttggcgagtagtggtc

SEQ ID NO: 12 - DNA, 16 bp, Artificial Sequence
  Synthetic oligonucleotide
acgaccgacgcgaacg

SEQ ID NO: 13 - DNA, 28 bp, Artificial Sequence
  Synthetic oligonucleotide
ctaacgtataacgaaaatcgtaacaacc

SEQ ID NO: 14 - DNA, 25 bp, Artificial Sequence
  Synthetic oligonucleotide
agtatgaagggtaggaagaattcgg

SEQ ID NO: 15 - DNA, 27 bp, Artificial Sequence
  Synthetic oligonucleotide
gttatcgtcgtcgtcgtttttgttgtc

SEQ ID NO: 16 - DNA, 23 bp, Artificial Sequence
  Synthetic oligonucleotide
gacttccgaaaaacacaaaatcg

SEQ ID NO: 17 - DNA, 20 bp, Artificial Sequence
  Synthetic oligonucleotide
cgtttttggtgagcgtcgtc

SEQ ID NO: 18 - DNA, 21 bp, Artificial Sequence
  Synthetic oligonucleotide
cctcaactaatcgcggaaacg

SEQ ID NO: 19 - DNA, 25 bp, Artificial Sequence
  Synthetic oligonucleotide
tggtgatggaggaggtttagtaagt

SEQ ID NO: 20 - DNA, 27 bp, Artificial Sequence
  Synthetic oligonucleotide
aaccaataaaacctactcctcccttaa

SEQ ID NO: 21 - DNA, 34 bp, Artificial Sequence
  Synthetic oligonucleotide
ttaaaacttaacgaaactaaaccaacccgaccgt

SEQ ID NO: 22 - DNA, 37 bp, Artificial Sequence
  Synthetic oligonucleotide
cctcgatacctactaactaactcgctcgctcaaacct

SEQ ID NO: 23 - DNA, 40 bp, Artificial Sequence
  Synthetic oligonucleotide
attaaaccgcaccaaaaaaactacctaaaaaaaacgaaaa

SEQ ID NO: 24 - DNA, 18 bp, Artificial Sequence
  Synthetic oligonucleotide
acccgaccccgaaccgcg

SEQ ID NO: 25 - DNA, 19 bp, Artificial Sequence
  Synthetic oligonucleotide
aactcgctcgcccgccgaa

SEQ ID NO: 26 - DNA, 30 bp, Artificial Sequence
  Synthetic oligonucleotide
ccttacctctaaataccaaccccaaacccg

SEQ ID NO: 27 - DNA, 26 bp, Artificial Sequence
  Synthetic oligonucleotide
cgttttgaggttcgggtttcgtcgtt

SEQ ID NO: 28 - DNA, 29 bp, Artificial Sequence
  Synthetic oligonucleotide
ccgaacaacgaactactaaacatcccgcg

SEQ ID NO: 29 - DNA, 28 bp, Artificial Sequence
  Synthetic oligonucleotide
aaaaacctctacgccccgcgattaacta

SEQ ID NO: 30 - DNA, 30 bp, Artificial Sequence
  Synthetic oligonucleotide
accaccacccaacacacaataacaaacaca

SEQ ID NO: 31 - DNA, 16 bp, Artificial Sequence
  Synthetic oligonucleotide
gcggtcggaaggggtc

SEQ ID NO: 32 - DNA, 18 bp, Artificial Sequence
  Synthetic oligonucleotide
actcgttcccgacgctcg

SEQ ID NO: 33 - DNA, 35 bp, Artificial Sequence
  Synthetic oligonucleotide
ttaaaactttaacgaaactaaaccaacccgaccgt

SEQ ID NO: 34 - DNA, 21 bp, Artificial Sequence
  Synthetic oligonucleotide
ataatgcgcgtggtgcagatc

SEQ ID NO: 35 - DNA, 21 bp, Artificial Sequence
  Synthetic oligonucleotide
ttgcagccgaggaagaagatg

(19) United States
(12) Patent Application Publication
(10) Pub. No.: US 2008/0299566 A1
(43) Pub. Date: Dec. 4, 2008

(54) Methylation of Gene Promoters as a Predictor of Effectiveness of Therapy

(76) Inventors: Stephen J. Meltzer, Lutherville, MD (US); James P. Hamilton, Baltimore, MD (US); Fumiaki Sato, Kyoto (JP)

Correspondence Address: SUGHRUE MION, PLLC, 2100 PENNSYLVANIA AVENUE, N.W., SUITE 800, WASHINGTON, DC, 20037, US

(21) Appl. No.: 12/097,427

(22) PCT Filed: Dec. 18, 2006
(86) PCT No.: PCT/US06/62242

§ 371 Date: Jun. 13, 2008

(51) Int. Cl. C12Q 1/68

(52) U.S. Cl. 435/6

(57) ABSTRACT

The present invention provides methods for identifying, diagnosing, evaluating or monitoring a disease state in a subject comprising identifying the methylation status of a panel of genes in the subject. The present invention also relates to identifying, diagnosing, evaluating or monitoring the responsiveness of a subject to a therapeutic regimen, with the methods comprising determining the methylation status of a panel of genes in the subject.

### CROSS REFERENCE TO RELATED APPLICATIONS

This application claims priority to U.S. Provisional Patent Application No. 60/750,811, filed 16 Dec. 2005, which is hereby incorporated by reference.

### STATEMENT REGARDING FEDERALLY SPONSORED RESEARCH OR DEVELOPMENT

Part of the work performed during development of this invention utilized U.S. Government funds under NIH, Grant No. CA85069, CA98450, CA01808, DK067872, and CA106763. The U.S. Government has certain rights in this invention.

### FIELD OF THE INVENTION

The present invention provides methods for identifying, diagnosing, evaluating or monitoring a disease state in a subject comprising identifying the methylation status of a panel of genes in the subject. The present invention also relates to identifying, diagnosing, evaluating or monitoring the responsiveness of a subject to a therapeutic regimen, with the methods comprising determining the methylation status of a panel of genes in the subject.

### BACKGROUND OF THE INVENTION

Esophageal cancer is the eighth most common malignancy and sixth most common cause of cancer death in the world. (See Parkin, D. M., CA Cancer J Clin, 55: 74-108, (2005)). Based upon molecular pathogenesis studies of esophageal cancer, there is a growing body of evidence for abnormal methylation of DNA as an early event in carcinogenesis. Specifically, methylation of the promoter regions of tumor suppressor genes is commonly found in many human malignancies, including esophageal carcinoma. (See Schulmann, K. et al., Oncogene, 24:4138-4148 (2005)). Methylation of these tumor-suppressor genes leads to the reduced expression of tumor suppressor genes, resulting in unchecked cellular growth, tissue invasion, angiogenesis, and metastases. (See Das, P. M. and Singal, R. J Clin Oncol, 22: 4632-4642 (2004) and Momparler, R. L. Oncogene, 22: 6479-6483 (2003)). Multiple studies have shown that promoter methylation of tumor suppressor genes may also underlie carcinogenesis. (See Eads, C. A., et al., Cancer Res., 61:3410-3418 (2001), Sato, F. et al. Cancer Res., 62: 6820-6822 (2002) and Takahashi, T., et al., Int. J. Cancer, 115:503-510 (2005), all of which are incorporated by reference). For example, Reprimo is frequently methylated in multiple human malignancies, including esophageal cancer (See Hamilton J P. et al., Clin. Cancer Res. 12:6637-6642 (2006), Sato N. et al., Cancer Res. 63:3735-3742 (2003), Wong T. S. et al., Int. J. Cancer, 117:697 (2005), Suzuki M. et al., Lung Cancer, 47:309-314 (2005), Takahashi T. et al., Int. J. Cancer, 115:503-510 (2005), all of which are hereby incorporated by reference). Recent evidence demonstrates that methylation of Reprimo occurs significantly more frequently in pre-cancerous Barrett's esophagus and adenocarcinoma of the esophagus than in normal esophagus or squamous cell cancer of the esophagus, suggesting that this epigenetic alteration is a specialized columnar cell-specific, early event with potential as a biomarker for the early detection of esophageal neoplasia (Hamilton J P et al. (2006)).

Reprimo is a cytoplasmic protein belonging to a family of molecules controlled by p53 that inhibit cell-cycle progression (See Hollstein M. et al., Science, 253:49-53 (1991), which is hereby incorporated by reference). p53, the tumor suppressor gene, is the most commonly mutated gene in human cancer (See Levine A. J. Cell, 116:S67-S69, 1 p following S9 (2004) and Gottlieb T. M. et al., Biochim. Biophys. Acta, 1287:77-102 (1996), which are both hereby incorporated by reference). In healthy cells, upon exposure to genotoxic agents or other noxious particles and stresses, the p53 protein is activated, resulting in abrogation of the cell cycle (See Sherr C. J. Cancer cell cycles. Science 274:1672-1677 (1996), Levine A. J., Cell 88:323-331 (1997) and el-Deiry W. S., Semin. Cancer Biol. 8:345-357 (1998), all of which are hereby incorporated by reference. This arrest in growth allows for coordination of cellular repair mechanisms and permits the organism to eliminate damaged cells. (el-Deiry W. S., (1998)).

The function of p53 is mediated primarily through activation of target genes (See Yu J. et al., Proc Natl. Acad. Sci. U.S.A., 96: 14517-14522 (1999) and Taylor W. R. et al., Oncogene, 20:1803-1815 (2001), both of which are hereby incorporated by reference). Indeed, previous research has demonstrated that expression of Reprimo is dependent upon p53 (See Casson A. G. et al., Cancer Res. 51:4495-4499 (1991), and that overexpression of Reprimo leads to arrest at the G2 phase of the cell cycle (See Ohki R. et al., J. Biol. Chem., 275: 22627-22630.1 (2000), which is hereby incorporated by reference). Furthermore, in a murine model of uterine sarcoma, Reprimo was significantly increased in normal uteri of p53 wildtype mice, but Reprimo expression was not increased in either normal uteri or in uterine sarcomas of p53 knockout animals (See Hollstein M et. al., Science, 253:49-53 (1991), which is hereby incorporated by reference).

Finally, reduced expression of p53 is common in patients with esophageal cancer (See Bennett W. P. et al, Cancer Res. 52:6092-6097 (1992), Cameron A. J., Mayo Clin. Proc. 73:457-461 (1998), and Jankowski J. A. et al., Am. J. Pathol. 154:965-73 (1999), all of which are hereby incorporated by reference). Thus, it appears that p53 and Reprimo are closely linked in pathways leading toward apoptosis, and that derangements in the functions of either gene are likely to constitute primary carcinogenic events as well as strong candidate markers of disease progression.

Despite the abundance of evidence that characterize certain events in esophageal cancer initiation, promotion and progression, the incidence of esophageal cancer in the United States is rising. Indeed, it is estimated that approximately 15,000 new cases of esophageal cancer were diagnosed in the year 2005. (See American Cancer Society. Cancer Facts & Figures. Atlanta, Ga.: American Cancer Society (2005)). Although patients with localized esophageal cancer may benefit from concomitant radiation and chemotherapy (Walsh, T. N., et al., N Engl J Med, 335:462-467 (1996)), these regimens are extremely grueling and may lead to many complications, including mucositis, pancytopenia, infection, frequent clinic visits and hospital admissions, and, in some cases, death. (See Walsh, T. N., et al., N Engl J Med, 335:462-467 (1996) and Wang, K. K., et al., Gastroenterology, 128:1468-1470 (2005), which are incorporated by reference). Moreover, despite recent advances in treatment, five-year survival rates are dismal (less than 20%). (See Shaheen, N. J., Gastroenterology, 128:1554-1566 (2005)). It is clear that new techniques, markers, and medicines are needed to diagnose, stratify, and treat patients with esophageal cancer.

Aberrant methylation across panels of genes correlates with prognosis of many cancers. (See Darnton, S. J., et al., Int J Cancer, 115:351-358 (2005), Kawakami, K., et al., J Natl Cancer Inst, 92:1805-1811 (2000), Kikuchi, S., et al., Clin Cancer Res, 11:2954-2961 (2005) and Catto, J. W., et al., J Clin Oncol, 23:2903-2910 (2005), all of which are incorporated by reference). Indeed, prior studies have validated analyzing methylation patterns across a panel of genes to predict prognosis in esophageal and rectal cancer. (See Brock, M. V., et al., Clin Cancer Res, 9:2912-2919 (2003), Ghadimi, B. M., et al., J Clin Oncol, 23:1826-1838 (2005), both of which are hereby incorporated by reference). Further, it has recently been demonstrated methylation of Reprimo predicts a poor response to chemotherapy and radiation in esophageal cancer. (See Hamilton J. P. et al., Clin Gastroenterol Hepatol. 4:701-708 (2006)). Methylation of promoter regions of other tumor suppressor genes may also predict a poor response to such therapies in esophageal cancer and other cancers.

### SUMMARY OF THE INVENTION

The present invention provides methods for predicting the responsiveness of a therapeutic regimen in a subject in need thereof, with the methods comprising determining the methylation status of a panel of genes in a test subject and using this methylation status of the panel of genes in a test subject to indicate whether the test subject will respond to the therapeutic regimen in question. Methylation status is indicative of the test subject's response to the therapeutic regimen in question.

The present invention also provides methods of customizing a therapeutic regimen for a subject in need thereof, with the methods comprising determining the methylation status of a panel of genes in a test subject and using the methylation status of the test subject to dictate a therapeutic regimen. Based upon said test subject's methylation status, a health care provider can then determine an appropriate therapeutic regimen going forward.

The present invention also provides methods of monitoring the progression of a disease state in a subject, with the methods comprising determining the methylation status of a panel of genes in a test subject at a first and second time point to determine a difference in methylation status in the panel of genes in the subject over time. A difference in methylation status in the panel of genes in the subject over time is indicative of the progression of said disease state.

The present invention also provides methods of diagnosing a disease state in a subject suspected of having a disease, with the methods comprising determining the methylation status of a panel of genes in a test subject and using the test subject's methylation status to indicate the presence of a disease state in the subject.

The present invention also provides methods of predicting the reoccurrence of a previously diagnosed disease state in a subject, with the methods comprising determining the methylation status of a panel of genes in a test subject and using the test subject's methylation status as predictive of the reoccurrence probability of a previously diagnosed disease state.

### BRIEF DESCRIPTION OF THE DRAWINGS

FIG. 1 depicts the standardized combined methylation-specific PCR (MSP) values in responders versus non-responders. The genes included in the panel are p16, p57, p73, CHFR, TIMP-3, MGMT, HPP1, runx-3, and/or Reprimo.

FIG. 2 depicts the percentage of methylated genes, as determined by MSP, in both responders and non-responders (NR) for each of the genes of a panel.

FIG. 3 depicts mean normalized methylation values (NMVs) obtained from quantitative Methylation-Specific PCR (qMSP) results for human normal esophagus (NE), Barrett's esophagus (BE), Barrett's esophagus with high-grade dysplasia (HGD); adenocarcinoma of the esophagus (EAC), squamous cell carcinoma of the esophagus (ESCC) tissues.

FIG. 4 depicts the mean NMV of Reprimo in normal esophagus (NE), short-segment Barrett's (SS BE), long-segment Barrett's (LS BE), high-grade dysplasia (HGD), and adenocarcinoma (EAC).

FIG. 5 depicts a receiver-operator curve (ROC) of NMVs of EAC versus NE.

FIG. 6 depicts NMVs obtained from qMSP results for Reprimo in ESCC cell lines.

FIG. 7 depicts NMVs obtained from qMSP results for Reprimo in EAC cell lines.

FIG. 8 depicts results of EAC cell line OE33 treatment with 5-Aza-2′-Deoxycytidine (5AzaC) begun after 24 hours of cell growth.

FIG. 9 depicts results of ESCC cell line KYSE 110 treatment with 5AzaC begun after 24 hours of cell growth.

### DETAILED DESCRIPTION OF THE INVENTION

The present invention provides methods for predicting the responsiveness of a subject to a therapeutic regimen. As used herein, “predicting” indicates that the methods described herein are designed to provide information to a health care provider or computer, to enable the health care provider or computer to determine the likely effectiveness of a proposed therapeutic regimen for the subject. Examples of health care providers include but are not limited to, an attending physician, oncologist, physician's assistant, pathologists, laboratory technician, etc. The information may also be provided to a computer, where the computer comprises a memory unit and machine executable instructions that are configured to execute at least one algorithm designed to determine the likely effectiveness of a proposed therapeutic regimen for the subject. Accordingly, the invention also provides devices for predicting the responsiveness of a subject to a therapeutic regimen comprising a computer with machine executable instructions for predicting the responsiveness of a subject to a therapeutic regimen.

As used herein, the term “subject” is used interchangeably with the term “patient,” and is used to mean an animal, in particular a mammal, and even more particularly a non-human or human primate.

As used herein, a “therapeutic regimen” is a plan for treating a subject in need of treatment for a particular disease state. Furthermore, the term “treat” is used to indicate a procedure which is designed to ameliorate one or more causes, symptoms, or untoward effects of an abnormal condition in a subject. The therapeutic regimen can, but need not, cure the subject, i.e., remove the cause(s), or remove entirely the symptom(s) and/or untoward effect(s) of the abnormal condition in the subject. More particularly, the phrase “therapeutic regimen” is also used to indicate a procedure which is designed to inhibit growth and accelerate cell aging, induce apoptosis and cell death of neoplastic tissue within a subject. Additionally, “therapeutic regimen” means to reduce, stall, or inhibit the growth of or proliferation of tumor cells, including but not limited to carcinoma cells. The therapeutic regimen may or may not be employed prior to performing the methods of the present invention. The invention is not limited by the therapeutic regimen contemplated. Examples of therapeutic regimens include but are not limited to chemotherapy (pharmaceuticals), radiation therapy, surgical intervention, cell therapy, stem cell therapy, gene therapy and any combination thereof. In one embodiment, the therapeutic regimen comprises chemotherapy. In another embodiment, the therapeutic regimen comprises radiation therapy. In yet another embodiment, the therapeutic regimen comprises surgical intervention. In still another embodiment, the therapeutic regimen comprises a combination of chemotherapy and radiation therapy.

Of course, the therapeutic regimen that is being employed or contemplated will depend on the abnormal condition that the subject has or is suspected of having. As used herein, an “abnormal condition” is used to mean a disease, or aberrant cellular or metabolic condition. Examples of abnormal conditions in which the methods can be used include but are not limited to, dysplasia, neoplastic growth and abnormal cell proliferation. In one embodiment, the abnormal condition comprises neoplastic growth. In a more specific embodiment, the abnormal condition comprises a carcinoma. In an even more specific embodiment, the abnormal condition comprises either squamous cell carcinoma or adenocarcinoma. The invention is not limited to the type of neoplasm or carcinoma. For example, the carcinoma may be a carcinoma of the digestive track or any associated glands or organs, including, but not limited to, the throat, the salivary glands, esophagus, the stomach, the small intestine, the large intestine, the pancreas, liver, gallbladder, biliary tree, and rectum. Additional forms of cancer include, but are not limited to, lung cancer, prostate cancer and breast cancer.

The methods comprise determining the methylation status of a panel of genes in the test subject. As used herein, “methylation status” is used to indicate the presence or absence or the level or extent of methyl group modification in the polynucleotide of at least one gene. As used herein, a “panel of genes” is a collection of genes comprising 3 or more distinct genes. In one embodiment, the panel of genes comprises at least 3, 4, 5, 6, 7, 8, 9, 10, 11, 12, 13, 14, 15, 16, 17, 18, 19 or 20 or more genes.

The term “gene” is used similarly to as it is in the art. Namely, a gene is a region of DNA that is responsible for the production and regulation of a polypeptide chain. Genes include both coding and non-coding portions, including introns, exons, promoters, initiators, enhancers, terminators and other regulatory elements. As used herein, “gene” is intended to mean at least a portion of a gene. Thus, for example, “gene” may be considered a promoter for the purposes of the present invention. Accordingly, in one embodiment of the present invention, at least one member of the panel of genes comprises a non-coding portion of the entire gene. In a particular embodiment, the non-coding portion of the gene is a promoter. In another embodiment, all members of the entire panel of genes comprise non-coding portions of the genes. In another particular embodiment, the non-coding portions of the members of the genes are promoters.

Candidate members of the gene panel include, but are not limited to, tumor suppressor genes, tumor promoter genes and other genes that may be involved in cell cycle regulation. Examples of genes involved in the regulation of cell cycle that could serve as members of the gene panel include, but are not limited to, Reprimo, p14, p15, p16, p27 and CHFR. The tumor genetics of p16 have been evaluated extensively, and its silencing can occur via mutation, loss of heterozygosity (LOH), homozygous deletion, or promoter hypermethylation. In addition, p16 is a member of the cyclin dependent kinase inhibitor (CDKI) family of genes and causes cell cycle arrest at the G1/S phase. p16 inactivation can result in uncontrolled cell growth. The product of the CHFR gene is responsible for a delay in chromosomal condensation during prophase in response to microtubule injury. Reprimo is a regulator of the p53-mediated cell cycle arrest point at G2/M. Other genes involved in cell cycle regulation will be recognized and appreciated by one of skill in the art.

Other candidate members of genes that may serve as members of the gene panel include, but are not limited to genes involved in angiogenesis. Examples of genes involved in angiogenesis include but are not limited to TIMP-1, TIMP-2, TIMP-3, TIMP-4, VEGF-A, VEGF-B, VEGF-C, VEGF-D, VEGF-E, IL-8, TGFβ and TGFα to name a few. One of skill in the art can recognize and appreciate genes involved in angiogenesis.

Still other candidate member genes include, but are not limited to genes involved in repair. Example of repair genes include, but are not limited to MGMT, BRCA1, BRCA2, hMLH1, hMSH1, hMLH6, and SHFM1 to name a few. One of skill in the art can recognize and appreciate gene repair genes.

Additional candidate genes include, but are not limited to genes encoding receptors, growth factors and transcription factors to name a few. Some examples of a candidate for gene to serve on the panel include, but are not limited to, Hpp-1, sVEGFR-2 (sFLK-1), IGFIR, IGFR, c-KIT, PDGFRα, HGFR, Grb2, bFGFR-2, FGFR-2, FGFR-3, PDEGF, RARBeta, and RASSF1A.

In one embodiment, the panel of gene comprises a combination of at least 3, 4 or 5 of the genes selected from the group consisting of Reprimo, p16, TIMP-3, MGMT, Hpp-1, and CHFR. In another embodiment, the panel of genes comprises the Reprimo, p16, TIMP-3, MGMT, Hpp-1 and the CHFR genes.

The invention is not limited by the types of assays used to assess methylation status of the members of the gene panel. Indeed, any assay that can be employed to determine the methylation status of the gene panel should suffice for the purposes of the present invention. In general, assays are designed to assess the methylation status of individual genes, or portions thereof. Examples of types of assays used to assess the methylation pattern include, but are not limited to, Southern blotting, single nucleotide primer extension, methylation-specific polymerase chain reaction (MSPCR), restriction landmark genomic scanning for methylation (RLGS-M) and CpG island microarray.

The measure of the levels of methylation a qualitative component or it may be quantitative. For example, the methylation status of a panel of genes may simply be considered, on the whole, as methylated or unmethylated, or the methylation status may be quantified is some numerical expression, such as a ratio or a percentage. Furthermore, the methylation status of each individual member of the panel of genes may be assessed, or the methylation status of the panel, as a whole, may be assayed, determined or considered.

The methylation status of the subject may be assessed in vivo or in vitro, from a sample from the subject. The samples may or may not have been removed from their native environment. Thus, the portion of sample assayed need not be separated or removed from the rest of the sample or from a subject that may contain the sample. Of course, the sample may also be removed from its native environment. For example, the sample may be a tissue section or body fluid, such as, but not limited to blood, plasma serum, cerebrospinal fluid, bile, urine, semen, synoviel fluid, sputum, saliva, and lymph. Furthermore, the sample may be processed prior to being assayed. For example, the sample may be diluted or concentrated; the sample may be purified and/or at least one compound, such as an internal standard, may be added to the sample. The sample may also be physically altered (e.g., centrifugation, affinity separation) or chemically altered (e.g., adding an acid, base or buffer, heating) prior to or in conjunction with the methods of the current invention. Processing also includes freezing and/or preserving the sample prior to assaying.

Once the methylation status of the panel of genes has been determined, this determination is then used to predict, indicate, or otherwise assess the responsiveness by a test subject to the therapeutic regimen in question. As used herein a subject that is or was responsive, a responder subject, is used to indicate that a therapeutic regimen was successful in detectably treating the subject. As used herein, predict means to provide an indicia of whether a particular treatment or therapeutic regimen will be successful. As used herein, indicate means to provide a basis to a health care practitioner whether a particular therapeutic regimen or treatment will be successful.

The methylation status of the test subject's panel of genes may be compared to one or more responding subjects, including, but not limited to a population of responding subjects. Or the methylation status of the test subject's panel of genes may be compared to one or more non-responding subjects, including, but not limited to a population of non-responding subjects. In addition, the methylation status of the test subject may be compared to his or her own previously assessed methylation status. Also, the methylation status of the test subject may be used to determine or assess the responsiveness of a therapeutic regimen.

A difference between the test subject's methylation status between two time points is an indication that the test subject may or may not respond to the therapeutic regimen in question. For example, a methylation status in the test subject at a first time point that is greater than the methylation status of the test subject at a second time point may indicate that the test subject may respond to the therapeutic regimen in question, whereas the test subject (at time point one) was predicted to not be responsive to the therapeutic regimen in question. Alternatively, a methylation status in the test subject that is lower at a first time point than the methylation status in the test subject at a second time point may indicate that the test subject may not respond at the second time point to the therapeutic regimen in question.

The present invention also provides methods of customizing a therapeutic regimen for a subject in need thereof, with the methods comprising determining the methylation status of a panel of genes in a test subject and using the methylation status of the test subject to dictate an appropriate therapeutic regimen going forward or indicate the responsiveness of a particular therapeutic regimen going forward.

The present invention also provides methods of monitoring the progression of a disease state in a subject, with the methods comprising determining the methylation status of a panel of genes in a test subject at a first and second time point to determine a difference in methylation status is the panel of genes in the subject over time. A difference in methylation status in the panel of genes in the subject over time is indicative of the progression of said disease state.

As used herein, the phrase “monitor the progression” is used to indicate that the abnormal condition in the subject is being periodically checked to determine if the abnormal condition is progression (worsening), regressing (improving) or remaining static (no detectable change) in the individual by assaying the methylation status in the subject using the methods of the present invention. The methods of monitoring may be used in conjunction with other monitoring methods or other treatments for the abnormal condition to monitor the efficacy of the treatment. Thus, “monitor the progression” is also intended to indicate assessing the efficacy of a treatment regimen by periodically assessing the methylation status of the panel of genes and correlating any differences in methylation status in the subject over time with the progression, regression or stasis of the abnormal condition. Monitoring may include two time points from which a sample is taken, or it may include more time points, where any of the methylation status at one particular time point from a given subject may be compared with the methylation status in the same subject, respectively, at one or more other time points.

The present invention also provides methods of diagnosing a disease state in a subject suspected of having a disease, with the methods comprising determining the methylation status of a panel of genes in a test subject and using the test subject's methylation status to indicate the presence of a disease state in the subject.

As used herein, the term “diagnose” means to confirm the results of other tests or to simply confirm suspicions that the subject may have an abnormal condition, such as cancer. A “test,” on the other hand, is used to indicate a screening method where the patient or the healthcare provider has no indication that the patient may, in fact, have an abnormal condition and may also be used to assess a patient's likelihood or probability of developing a disease or condition in the future. The methods of the present invention, therefore, may be used for diagnostic or screening purposes. Both diagnostic and testing can be used to “stage” the abnormal condition in a patient. As used herein, the term “stage” is used to indicate that the abnormal condition or obesity can be categorized, either arbitrarily or rationally, into distinct degrees of severity. The term “stage,” however, may or may not involve disease progression. The categorization may be based upon any quantitative characteristic or be based upon qualitative characteristics that can be separated. An example of staging includes but is not limited to the Tumor, Node, Metastasis System of the American Joint Committee on Cancer. For example, in esophageal cancer, in stage T1 of esophageal cancer, the tumor is only in the lining of the esophagus. In stage T2, the tumor has moved into the layer of muscles in the esophageal wall. In stage T3, the tumor has advanced through the entire esophageal wall. And in stage T4, the tumor has affected nearby tissues.

The present invention also provides for kits for performing the methods described herein. Kits of the invention may comprise one or more containers containing one or more reagents useful in the practice of the present invention. Kits of the invention may comprise containers containing one or more buffers or buffer salts useful for practicing the methods of the invention. A kit of the invention may comprise a container containing a substrate for an enzyme, a set of primers and reagents for PCR, etc.

Kits of the invention may comprise one or more computer programs that may be used in practicing the methods of the invention. For example, a computer program may be provided that calculates a methylation status in a sample from results of the detecting levels of antibody bound to the biomarker of interest. Such a computer program may be compatible with commercially available equipment, for example, with commercially available microarray or real-time PCR. Programs of the invention may take the output from microplate reader or realtime-PCR gel and prepare a calibration curve from the optical density observed in the wells or gel and compare this densitometric reading to the optical density readings in wells with test samples.

### EXAMPLE 1

Endoscopic biopsies were obtained from the esophageal tumors of thirty-five untreated patients who were consecutively enrolled in a treatment protocol at the University of Maryland, Baltimore. The Institutional Review Board and the Office of Research on Human Subjects at the University of Maryland, Baltimore, approved the Marlene and Stewart Greenebaum Cancer Center treatment protocol #9967.

The clinical characteristics of the patients can be found in Table I. Tumor samples were immediately frozen on dry ice and stored at −80° C. Diagnosis of the tumors was verified via histological methods. After staging, each patient received two cycles of induction chemotherapy with cisplatin (75 mg/m²/day) and 5-fluorouracil (1000 mg/m²/day.), coupled with concurrent x-ray radiation (56.4 Gy). One month after induction chemotherapy, the treated patients were re-staged using esophagogastroduodenoscopy (EGD), computer tomography (CT) scans of the chest and abdomen, and positron emission tomography (PET) scans. After re-staging, patients underwent esophagectomy. The surgical resection was examined for the presence or absence of tumor, and, in most cases (32/35), response to chemotherapy and radiation was determined after surgery. If no tumor was detectable in the esophagectomy specimen, the patient was defined as a responder (R). If tumor was present in the specimen, the patient was defined as a non-responder (NR). In addition if metastases or other indications of disease progression were discovered during re-staging, the patient was defined as a non-responder.

TABLE I

| Variable | n = 35 |
|---|---|
| Age, y, mean (range) | 61 (37-81) |
| Race (Caucasian/African American) | 32/3 |
| Sex (m/f) | 28/7 |
| UICC Stage IIa | 1 |
| UICC Stage IIb | 2 |
| UICC Stage III | 32 |
| Adenocarcinoma | 22 |
| Squamous Cell Carcinoma | 13 |

Gene Selection—Eleven candidate genes were selected based on their known ability to predict responsiveness to chemoradiation and prognosis in esophageal cancer, or upon their role in governing cell cycle. The G2/M phase of the cell cycle, in particular, was targeted because cells in the G2/M phase are most sensitive to X-ray-induced apoptosis (See Radford, I. R., Int J Radiat Biol, 65:203-215 (1994) and Radford, I. R., et al., Int J Radiat Biol, 65:217-227 (1994), Shinomiya, N. J Cell Mol Med, 5:240-253 (2001), all of which are incorporated by reference). In addition, genes were selected based on their known involvement in human carcinogenesis. Specifically, Reprimo (the Greek word for “repress”) is a mediator of p53-mediated cell cycle arrest at the G2/M phase. (See Ohki, R., et al., J Biol Chem, 275:22627-22630 (2000), incorporated by reference). Reprimo is frequently methylated in a variety of human malignancies and is also induced by X-irradiation. (See Takahashi, T., et al., Int J Cancer, 115:503-510 (2005), incorporated by reference). O⁶-methylguanine-DNA methyltransferase (MGMT,) a DNA excision repair gene, is commonly methylated in esophageal cancer, (Eads, C. A., et al., Cancer Res, 61:3410-3418 (2001)), and promoter hypermethylation of MGMT has been correlated with a response to alkylating agents in brain tumors. (See Esteller, M., et al., N Engl J Med, 343:1350-1354, (2000), incorporated by reference). Similarly, CHFR (checkpoint with fork-head associated and ring finger) exists as part of an early G2/M checkpoint (Kang, D., et al., J Cell Biol, 156:249-259 (2002), incorporated by reference), and lack of expression of CHFR in esophageal cancer has been linked to promoter hypermethylation. (See Shibata, Y., et al., Carcinogenesis, 23:1695-1699 (2002), incorporated by reference). Tissue inhibitor of metalloproteinase-3 (TIMP-3) encodes a potent inhibitor of angiogenesis, and methylation of its promoter is associated with a poor prognosis in esophageal cancer. (See Darnton, S. J., et al., Int J Cancer, 115: 351-358 (2005), incorporated by reference). p16 and p57 belong to a family of cyclin-dependent kinase inhibitors that cause cell cycle arrest at the G1 phase. Methylation and subsequent lack of expression of p16 in esophageal cancer are also associated with a poor prognosis. (See Brock, M. V., et al., Clin Cancer Res, 9:2912-2919 (2003), incorporated by reference). Methylation of p57 has been reported in multiple human malignancies. (See Kobatake, T., et al., Oncol Rep, 12:1087-1092 (2004), incorporated by reference). Methylation of RUNX-3 (runt-related transcription factor 3) is observed in esophageal cancer and is associated with progression from Barrett's esophagus with low-grade dysplasia to Barrett's adenocarcinoma. (See Schulmann, K. et al., Oncogene, 24:4138-4148 (2005)). Methylation of HPP1 (hyperplastic polyposis) is also correlated with Barrett's—associated neoplastic progression. (Schulmann, K. et al., Oncogene, 24:4138-4148 (2005)). Methylation of HPP1 is found in esophageal, (Schulmann, K. et al., Oncogene, 24:4138-4148 (2005)), and gastric and colon cancers (See Shibata, D. M., et al., Cancer Res, 62:5637-5640 (2002), Young, J., et al., Proc Natl Acad Sci USA, 98:265-270 (2001) and Shibata, D., et al., Gastroenterology, 128:a-787 (2005), all of which are incorporated by reference). The exact function of HPP1 has not been determined, but it encodes an epidermal growth factor domain and is therefore thought to play a role in cell growth, maturation, and adhesion. (See Shibata, D. M., et al., Cancer Res, 62:5637-5640 (2002), Young, J., et al., Proc Natl Acad Sci USA, 98:265-270 (2001)).

The role of p73 in esophageal cancer is unclear, but it is a homologue of p53, and this family of genes functions as transcription factors that play a major role in regulating the response of mammalian cells to stressors and damage, in part through the transcriptional activation of genes involved in cell cycle control, DNA repair, senescence, angiogenesis and apoptosis. (See Maley, C. C., et al., Cancer Res, 64:7629-7633 (2004), Heeren, P. A., et al., Anticancer Res, 24:2579-2583 (2004), Souza, R. F., Surg Oncol Clin N Am, 11:257-272, viii (2002), Chiarugi, V., et al., Cell Mol Biol Res, 40:603-612 (1994) and Meikrantz, W. and Schlegel, R., J Cell Biochem, 58:160-174 (1995), all of which are incorporated by reference. Finally, XAF-1 (X-linked inhibitor of apoptosis-1) and cyclooxygenase-2 (COX-2) were selected because of their respective roles in regulating apoptosis, the cell cycle, and inflammatory responses (See Chawla-Sarkar, et al., Apoptosis, 8:237-249 (2003) and Akhtar, M., et al., Cancer Res, 61:2399-2403 (2001), both of which are incorporated by reference). The expression of XAF-1 has been linked to resistance to cisplatin in vitro, Yang, X., et al., Gynecol Oncol, 97: 413-421 (2005), incorporated by reference, and COX-2 expression has been correlated to responsiveness to radiation and chemotherapy in gynecologic squamous cell malignancies. (See Pyo, H., et al., Int J Radiat Oncol Biol Phys, 62:725-732 (2005) incorporated by reference).

Statistical Analysis—The normalized methylation value of the genes was compared in responders vs. non-responders using the Student's paired t-test (Statistica 6.0.) In addition, because many of the data points were equal to zero, further non-parametric analysis was performed on the methylation values of the genes using the Mann-Whitney U test (Statistica 6.0.) After a qualitative methylation status was assigned, the individual genes were tested for significance with regards to response to therapy using Fisher's exact test.

Pre-screening of candidate genes for methylation in normal white blood cells—Based upon the assumption that methylation of esophageal tumor suppressor genes should not occur in normal white blood cells (WBCs), candidate genes were tested for methylation in WBCs.

DNA Extraction and Quantitative Methylation Specific PCR (MSP)—DNA from the frozen tumor specimens were extracted using previously published protocols. (See Sato, F. et al. Cancer Res., 62: 6820-6822 (2002) and Meltzer, S. J., et al. Cancer Res., 54:3379-3382 (1994), which are hereby incorporated by reference). DNA methylation of Reprimo, p16, CHFR, MGMT, TIMP-3, HPP1, was determined by quantitative methylation specific PCR (MSP) using the Taqman system (Applied Biosystems, Foster City, USA) (Eads, C. A., et al., Cancer Res., 61: 3410-3418 (2001)). MSP distinguishes between methylated and unmethylated alleles of a given gene based on DNA sequence alterations after bisulfite treatment of DNA. Bisulfite treatment converts unmethylated but not methylated cytosines to uracils. Subsequent PCR using primers and probe specific to the corresponding methylated DNA sequence is then performed. β-Actin was selected as an internal control, and analysis was based on previously published primer and probe sequences (Eads, C. A. et al., (2001) and Sato, F., et al. (2002)). Bisulfite-treated DNA extracted from the white blood cells of normal patients was used as an additional negative control. Briefly, 1.0 μg of genomic DNA was denatured by treatment with NaOH and modified by sodium bisulfite. DNA samples were purified using Wizard DNA clean-up resin (Promega, Madison, USA), treated with NaOH, precipitated with ethanol, and re-suspended in 50 μl of water. The PCR mixture consisted of 12.5 μl of Taqman Universal Master Mix without UNG (Applied Biosystems), 2.0 μl of probe for both the gene of interest and β-Actin (2.5 μM), 0.25 μl of forward and reverse primer for both the gene of interest and β-Actin (10 μM), 50 ng of bisulfite treated DNA, and water (up to a total volume of 25 μl.) PCR and real-time data collection were performed using an ABI7700 Sequence Detection System (Applied Biosystems) for activation of Taq polymerase at 95° C. for 10 minutes and then 50 cycles consisting of denaturation at 95° C. for 15 seconds and annealing and extension for 1 minute at 60° C. CpG Universal Methylated DNA (Intergen, Burlington, USA) was used to generate a standard curve for each reaction. Reaction mix without any bisulfite-treated DNA served as a negative control (Eads, C. A., et al., Cancer Res., 61: 3410-3418 (2001)). The forward and reverse primers are displayed in Table II. Table III displays the sequences for forward and reverse primers for each gene used in the Methylation Specific PCR.

TABLE II
Forward and Reverse Primers

| | | |
|---|---|---|
| Reprimo | Frwd | 5′-CGC GTC GGA AGG GGT C-3′ |
| | | (SEQ ID NO. 1) |
| | Rev | 5′-ACT CGT TCC CGA CGC TCG-3′ |
| | | (SEQ ID NO. 2) |
| P57 | Frwd | 5′-CGT TTT ATA GGT TAA GTG CGT TGT |
| | | GTT C-3′ |
| | | (SEQ ID NO. 3) |
| | Rev | 5′-ATT GCG CTA TCT CGT CCG AAC G-3′ |
| | | (SEQ ID NO. 4) |
| P73 | Frwd | 5′-GTT CGG GAT TTC GAT TTG GAC-3′ |
| | | (SEQ ID NO. 5) |
| | Rev | 5′-CCA CCG AAT CGC GCA G-3′ |
| | | (SEQ ID NO. 6) |
| P16 | Frwd | 5′-TGGAATTTTCGGTTGATTGGTT-3′ |
| | | (SEQ ID NO. 7) |
| | Rev | 5′-AACAACGTCCGCACCTCGT-3′ |
| | | (SEQ ID NO. 8) |
| TIMP-3 | Frwd | 5′-GCGTCGGAGGTTAAGGTTGTT-3′ |
| | | (SEQ ID NO. 9) |
| | Rev | 5′-CTCTCCAAAATTACCGTACGCG-3′ |
| | | (SEQ ID NO. 10) |
| RUNX-3 | Frwd | 5′-GGGTTTTGGCGAGTAGTGGTC-3′ |
| | | (SEQ ID NO. 11) |
| | Rev | 5′-ACGACCGACGCGAAGG-3′ |
| | | (SEQ ID NO. 12) |
| MGMT | Frwd | 5′-CTAACGTATAACGAAAATCGTAACAACC-3′ |
| | | (SEQ ID NO. 13) |
| | Rev | 5′-AGTATGAAGGGTAGGAAGAATTCGG-3′ |
| | | (SEQ ID NO. 14) |
| Hpp-1 | Frwd | 5′-GTTATCGTCGTCGTCGTTTTTGTTGTC-3′ |
| | | (SEQ ID NO. 15) |
| | Rev | 5′-GACTTCGGAAAAACACAAAATCG-3′ |
| | | (SEQ ID NO. 16) |
| CHFR | Frwd | 5′-CGT TTT TGG TGA GCG TCG TC-3′ |
| | | (SEQ ID NO. 17) |
| | Rev | 5′-CCT CAA CTA ATC GCG GAA ACG-3′ |
| | | (SEQ ID NO. 18) |
| B-Actin | Frwd | 5′-TGGTGATGGAGGAGGTTTAGTAAGT-3′ |
| | | (SEQ ID NO. 19) |
| | Rev | 5′-AACCAATAAAACCTACTCCTCCCTTAA-3′ |
| | | (SEQ ID NO. 20) |

TABLE III
Methylation Specific PCR Probes

| | |
|---|---|
| Reprimo | 6FAM-TTA AAA CTT AAC GAA ACT AAA CCA ACC |
| | CGA CCG T-TAMRA |
| | (SEQ ID NO. 21) |
| P57 | 6FAM-GCT CGA TAC CTA CTA ACT AAC TCG CTC |
| | GCT CAA ACC T-TAMRA |
| | (SEQ ID NO. 22) |
| P73 | 6FAM-ATT AAA CCG GAG CAA AAA AAC TAG CTA |
| | AAA AAA ACG AAA A |
| | TAMRA (SEQ ID NO. 23) |
| P16 | 6FAM-FAM-ACCCGACCCCGAACCGCG-TAMRA |
| | (SEQ ID NO. 24) |
| TIMP-3 | 6FAM-AACTCGCTCGCCCGCCGAA-TAMRA |
| | (SEQ ID NO. 25) |
| MGMT | 6FAM-CCTTACCTCTAAATACCAACCCCAAACCCG-TAMRA |
| | (SEQ ID NO. 26) |
| RUNX-3 | 6FAM-CGTTTTGAGGTTCGGGTTTCGTCGTT6-TAMRA |
| | (SEQ ID NO. 27) |
| Hpp-1 | 6FAM-CCGAACAACGAACTACTAAACATCCCGCG-TAMRA |
| | (SEQ ID NO. 28) |
| CHFR | 6FAM-AAA AAC CTC TAC GCC CCG CGA TTA ACT |
| | A-TAMRA |
| | (SEQ ID NO. 29) |
| B-Actin | 6VIC-ACCACCACCCAACACACAATAACAAACACA-TAMRA |
| | (SEQ ID NO. 30) |

Analysis of MSP Results—The normalized MSP value (NMV) was calculated by dividing the ratio of the quantitative MSP value for the gene of the interest to β-actin for each sample by the ratio of the quantitative MSP value for the gene of interest to β-actin for Universal Methylated DNA. (Sato, F., et al. Cancer Res., 62: 6820-6822 (2002) and Shibata, D M., et al., Cancer Res., 62:5637-5640 (2002), incorporated by reference). The qualitative MSP status is determined by analyzing the normalized MSP value. A Normalized MSP value of 0.05 was assigned as the cutoff point for classifying a result a positive (≧0.05) or negative (≦0.05) methylation status. The cutoff point was determined by ROC curve analysis as has been published previously by Schulmann, K. et al., Oncogene, 24:4138-4148 (2005), which is hereby incorporated by reference.

Response to Combined Modality Treatment—Thirteen (37%) of the 35 patients were responders. Twenty-two (63%) of the 35 five patients were non-responders. Two of the 22 non-responders had evidence metastasis after therapy, and were removed as candidates for surgery.

Methylation Specific PCR—The XAF-1 and COX-2 genes were excluded from the study, because promoter methylation was detected in the white blood cell DNA from normal patients. Of the remaining genes, methylation of white blood cell control DNA was not detected. p57 and p73 were also excluded from the study, because of the low percentage of methylation amongst responders and non-responders. Specifically, methylation of p57 was found in 0 of 13 responders (0%) and only in 1 of the 22 non-responders (5%). Methylation of p73 was detected in 2 of the 22 non-responders (9%) and in none of the responders (0%).

In addition, RUNX-3 was excluded from further analysis, because its methylation pattern indistinguishable between responders and non-responders. Specifically, RUNX-3 was methylated in 5 of the 13 responders (38%) and in 7 of the 22 non-responders (32%).

Promoter hypermethylation of p16, CHFR, MGMT, TIMP-3, HPP1 and Reprimo was seen more frequently in non-responders than in responders. In this study, p16 was methylated in 8% of patients who did vs. 27% of patients who did not respond to treatment. In our study, CHFR was methylated in 32% of patients who did not respond but in only 8% of patients who did respond to the treatment of esophageal cancer. Methylation of MGMT was found in 41% of patients who did not respond but in only 23% of patients who did respond to chemotherapy and radiation. 45% of patients who did not respond to chemotherapy and radiation had methylated TIMP-3, whereas only 15% of patients who responded were methylated at TMIP-3. It was found that HPP1 is more frequently methylated in non-responders (50%) than in the responders (15%.). In the current study, nearly two-thirds (64%) of patients who did not respond vs. only 15% of patients who did respond to chemotherapy and radiation had Reprimo promoter methylation.

The qualitative MSP results for the individual genes are found in Table IV. The frequency of Reprimo methylation was significantly greater in non-responders than in responders (p=0.01). The normalized MSP values in non-responders compared to responders were also significantly different for the Reprimo promoter (Mann-Whitney U test p=0.037).

TABLE IV
Frequency and Mean Level⁺ of Methylation-Specific
PCR for Individual Genes

| | | | | | | Mean |
|---|---|---|---|---|---|---|
| | | | Mean | Non- | | MSP |
| Gene Name | Responders | | MSP (R) | responders | | (NR) |
| p57 | 0/13 | (0%) | 0.0004 | 1/22 | (5%) | 0.019 |
| Runx-3 | 5/13 | (38%) | 0.067 | 7/22 | (32%) | 0.090 |
| MGMT | 3/13 | (23%) | 0.059 | 9/22 | (41%) | 0.062 |
| p73 | 0/13 | (0%) | 0.0 | 3/22 | (14%) | 0.113 |
| p16 | 1/13 | (8%) | 0.031 | 6/22 | (27%) | 0.120 |
| CHFR | 1/13 | (8%) | 0.040 | 7/22 | (32%) | 0.068 |
| TIMP3 | 2/13 | (15%) | 0.022 | 10/22 | (45%) | 0.107 |
| HPP1 | 2/13 | (15%) | 0.085 | 11/22 | (50%) | 0.274 |
| Reprimo | 2/13 | (15%) | 0.078 | 14/22 | (64%)* | 0.313 |
| Total | 16/117 | (14%) | | 67/198 | (34%) | |

⁺Mean MSP values for each gene include data from all 35 patients.
*Fisher's exact test, p = 0.01

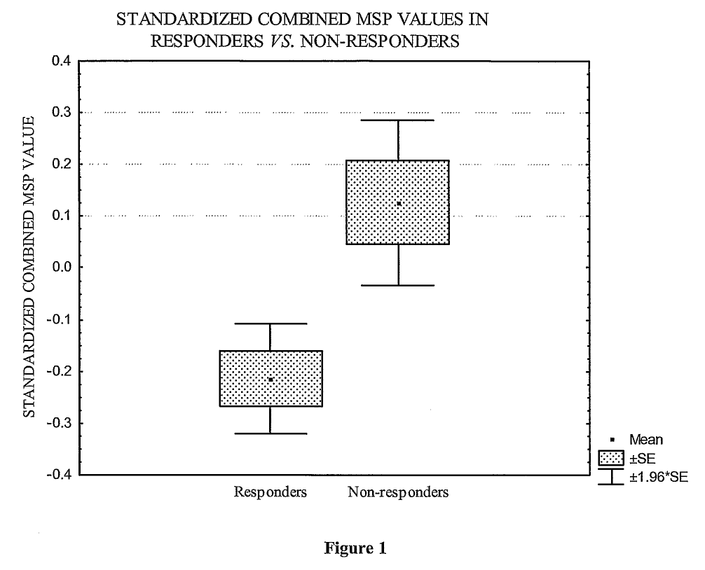

FIG. 1 shows that there was a significant difference between responders and non-responders when all the normalized MSP values of these 6 genes were analyzed. Specifically, the mean normalized MSP value for the panel of promoters (p16, CHFR, MGMT, TIMP-3, HPP1 and Reprimo) was 0.052 in the responders, and the mean normalized MSP value in non-responders for this panel was 0.157 (p=0.0007).

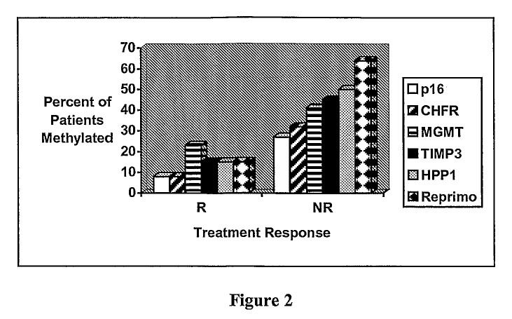

FIG. 2 shows the differences in percentages of patients methylated in responders vs. non-responders for the six genes showing the most marked differences in methylation between the two groups. As seen in FIG. 2, Reprimo displays significant difference in methylation between responders and non-responders. A normalized MSP value of 0.05 was assigned as the cutoff point for classifying methylation status as positive (≧0.05) or negative (<0.05).

### EXAMPLE 2

One hundred seventy-five esophageal samples were serially obtained endoscopically from 25 patients with Barrett's esophagus (BE), 45 with esophageal squamous cell cancer (ESCC), 75 with esophageal adenocarcinoma (EAC), 11 with high-grade dysplasia (HGD), and 19 with refractory gastroesophageal reflux symptoms but normal esophageal mucosa. The demographics of the patient subjects are displayed in Table V.

TABLE V
Patient Demographics

| Tissue | | | | | | | | |
|---|---|---|---|---|---|---|---|---|
| Type | n | Age (y) | Sex | | Race | | Other | |
| EAC | 75 | 63.5 ± 11.9 | 70 m | (93.3%) | 65 white | (86.6%) | UICC stage | |
| | | | 5 f | (6.6%) | 5 Asian | (6.6%) | I: 7 | (9.3%) |
| | | | | | 5 AA | (6.6%) | II: 16 | (21.3%) |
| | | | | | | | III: 35 | (46.6%) |
| | | | | | | | IV: 17 | (22.6%) |

| | | | | | | | |
|---|---|---|---|---|---|---|---|
| HGD | 11 | 70.3 ± 7.9 | 11 m | | 11 white | | |
| BE | 25 | 61.2 ± 14.7 | 24 m | (96%) | 20 white | (80%) | 9 short-segment |
| | | | 1 f | (4%) | 5 AA | (20%) | 16 long-segment |
| NE | 19 | 62.6 ± 11.5 | 14 m | (74.7%) | 18 white | (94.7%) | |
| | | | 5 f | (26.3%) | 1 AA | (5.3%) | |
| ESCC | 45 | 62.4 ± 7.8 | 33 m | (73.3%) | 28 white | (62.2%) | UICC stage |
| | | | 12 f | (26.7%) | 2 Asian | (4.5%) | not available |
| | | | | | 15 AA | (33.3%) | |

The samples consisted of 75 EAC, 45 ESCC, 25 BE, 11 HGD, and 19 NE. All patients were of similar age (Student's t-test, NE vs. BE, p=0.74; NE vs. HGD, p=0.06, NE vs. EAC, p=0.76; NE vs. ESCC p=0.96.) In all histologic types, the overwhelming majority of patients were white (NE, 94.7%; BE, 80%; HGD, 100%; EAC, 86.6%; ESCC, 62.2%,) and male (NE, 74.7%; BE, 96%; HGD, 100%; EAC, 93%; ESCC, 73.3%.)] According to Zhang Z., et al., Cancer Res. 62:3024-9 (2002), which is hereby incorporated by reference, BE was defined as long-segment if it was greater than or equal to 3 cm, and short-segment if less than 3 cm. In this study, there were 9 short-segment cases of BE and 16 long-segment cases of BE. Among the patients with EAC, there were 7 cases with UICC stage I disease, 17 with Stage II, 35 with stage III, and 16 with Stage IV disease. Tumor stage data was not available for all of the ESCC cases.

Samples were immediately frozen on dry ice and stored at −80° C. until DNA extraction. Tissue from cancers, BE, or NE was also sent for histology to confirm the pathologic diagnosis. The NE samples showed no endoscopic or microscopic evidence of premalignant or malignant lesions. Patients with BE had neither endoscopic nor microscopic evidence of dysplasia or tumor. A separate category of patients with BE had histologically confirmed HGD.

Cell Lines

Three esophageal adenocarcinoma cell lines (BIC, OE33, SEG) and eleven squamous cell carcinoma cell lines (KYSE 30, 70, 110, 140, 170, 180, 220, 410, 520, 770, 850) were obtained and stored at −80° C.

Primer and Probe Design

Primers and Probe for qMSP were designed based on the UCSC Human Genome Browser sequence data, and manufactured by Integrated DNA technologies (Coralville, Iowa). Probe and primer sequences are listed in Table VI.

TABLE VI
Probe and Primer Sequences for qMSP

| | |
|---|---|
| Probe and Primers | Sequences (5′→3′) |

Quantitative MSP

| | |
|---|---|
| Forward | GCGGTCGGAAGGGGTC |
| Reverse | ACTCGTTCCCGACGCTCG |
| Probe | TTAAAACTTTAACGAAACTAAACCAACCCGAC |
| | CGT |

Quantitative RT-PCR

| | |
|---|---|
| Forward | ATAATGCGCGTGGTGCAGATC |
| Reverse | TTGCAGCCGAGGAAGAAGATG |

DNA and RNA Extraction

DNA from frozen primary tissue specimens were extracted using previously published protocols (Meltzer et al., Cancer Res., 54:3379-82 (1994) and Sato F. et al., Cancer Res., 62:6820-2 (2002)). Briefly, cell line DNAs were purified with Proteinase K and extracted onto silica-gel membranes using DNeasy (Qiagen, Valencia, Calif.). Cell line RNA was isolated with phenol-chloroform and guanidine isothiocyanate according to the manufacturer's specifications (Trizol, Invitrogen, Carlsbad, Calif.) (See Chomczynski P. et al, Anal Biochem., 162:156-92 (1987), which is hereby incorporated by reference).

Quantitative Methylation-Specific PCR (qMSP)

DNA methylation of Reprimo was determined by qMSP using the ABI 7700 Taqman system (Eads C. A. et al., (2001)). MSP distinguishes methylated alleles of a given gene based on DNA sequence alterations after bisulfite treatment of DNA. Bisulfite treatment converts unmethylated but not methylated cytosines to uracils. Subsequent PCR using primers and probe specific to the corresponding methylated DNA sequence is then performed. (Eads C. A. et al., (2001)).

Analyses of MSP Results

The normalized MSP value (NMV) was calculated by dividing the ratio of the qMSP value for Reprimo to β-actin for each sample by the ratio of the qMSP value for Reprimo to, β-actin for Universal Methylated DNA (See Sato F. et al., Cancer Res., 62:6820-6822 (2002) and Shibata, D. M., et al., Cancer Res., 62:5637-5640 (2002)). Qualitative MSP status was determined by analyzing the normalized MSP value. A normalized MSP value of 0.05 was assigned as the cutoff point for classifying methylation status as qualitatively positive (≧0.05) or negative (<0.05). This cutoff point had been previously determined by ROC curve analysis (See Shibata, D. M., et al., (2002)).

MSP of Esophageal Tissues

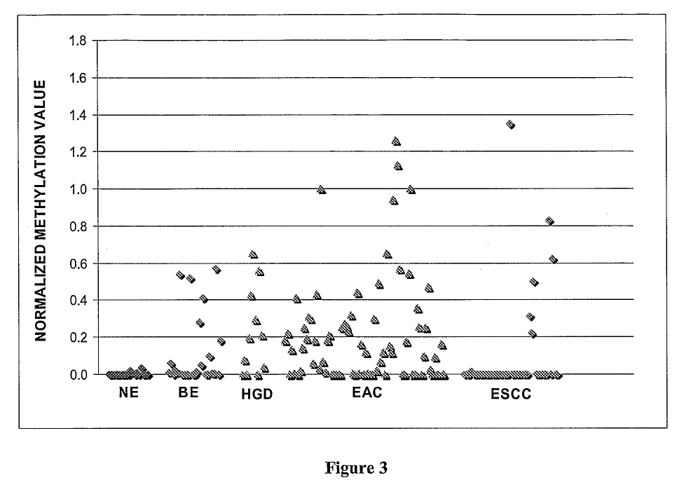

MSP results are displayed in FIG. 3 and Table VII. FIG. 3 shows that the mean normalized methylation values for each tissue type are: 0.004 for NE, 0.111 for BE, 0.222 for HGD, 0.249 for EAC, and 0.088 for SCCA. The differences between NE and BE, NE and HGD, and NE and EAC are significant (Student's-t test).

Reprimo methylation was significantly more common in BE, HGD, and EAC than in NE. In addition, within a set of patients with Barrett's, those with long-segment BE had significantly more Reprimo methylation than did those with short-segment disease (Student's t-test, p=0.048.) In fact, it was found that Reprimo methylation levels in EAC (0.249) were more than double their levels in BE (0.111), implying that they actually increased during progression from BE to EAC. Reprimo methylation levels in ESCC were not statistically different from those in NE. Thus, methylation of Reprimo may represent an early event that is critical for and unique to EAC. Methylation of other candidate genes described herein may also represent early events that are critical for the irritation and/or progression of EAC and other carcinomas.

TABLE VII
Normalized Quantitative Methylation Values and Qualitative
Methylation Status for Human Esophageal Tissues

| | | Student's t-test, | Mann-Whitney | Methylation | |
|---|---|---|---|---|---|
| Tissue Type | NMV | p= | test, p= | status (% of n) | |
| NE (n = 19) | 0.004 | n/a | n/a | 0 | (0%) |
| BE (n = 25) | 0.111 | 0.019 | 0.001 | 9 | (36%) |
| HGD (n = 11) | 0.223 | 0.003 | 0.001 | 7 | (64%) |
| EAC (n = 75) | 0.249 | 0.02 | 0.00003 | 47 | (63)% |
| SCCA (n = 45) | 0.088 | 0.171 | 0.67 | 6 | (13.3%) |

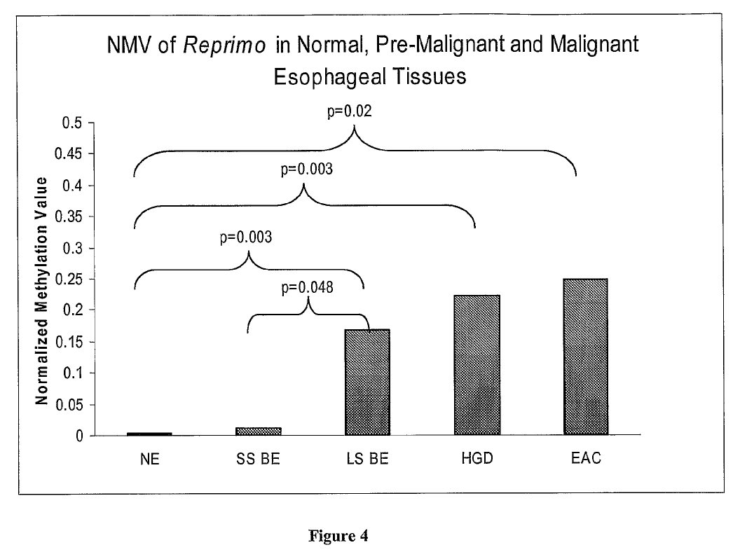

The difference in quantitative methylation of NE vs. BE was significant (Student's t test, p=0.02; Mann-Whitney's U test, p=0.001), as were the differences in quantitative methylation of NE vs. HGD (Student's t-test, p=0.003; Mann-Whitney's U test, p=0.001), and between NE and EAC (Student's t test, p=0.02; Mann-Whitney's U test, p=0.00003). Interestingly, there was a significant difference in the NMV for Reprimo between short-segment (mean NMV: 0.012) and long-segment (mean NMV: 0.168) BE (Student's t test, p=0.048). The mean NMVs for NE, short-segment BE, long-segment BE, HGD, and EAC are displayed in FIG. 4. The differences in mean NMV between NE and LS BE, SS BE, and LS BE, NE and HGD, as well as NE and EAC were significant (Student's t-test)

The differences between methylation levels of columnar tissues and ESCC were highly significant. The p-value of EAC vs. ESCC by Mann-Whitney testing was 0.000002, for HGD vs. ESCC 0.002, and for BE vs. ESCC 0.0001. The pathogenesis of EAC requires a coordinated accumulation of genomic and epigenetic abnormalities that is initiated by the chronic reflux of acidic fluid into the esophagus (See Jankowski J. A., et al., Am. J. Pathol., 154:965-973 (1999), Enzinger P. C. et al., N Engl J Med 349:2241-2252 (2003), and Montgomery E., et al., Hum. Pathol. 32:379-388 (2001), all of which are hereby incorporated by reference). Chronic reflux leads to the gradual replacement of normal squamous epithelium with specialized columnar cells, or Barrett's esophagus (Jankowski J. A., et al. (1999)). A small but significant portion of patients with BE will proceed to develop HGD and then EAC (See Weston A. P., et al., Am. J. Gastroenterol. 95:1888-1893 (2000) and Hage M. et al., Scand. J. Gastroenterol. 39:1175-1179 (2004), incorporated by reference). It is now apparent that the length of the Barrett's segment is an important predictor of this neoplastic progression (Wang S. et al., Oncogene 25:3346-3356 (2006).

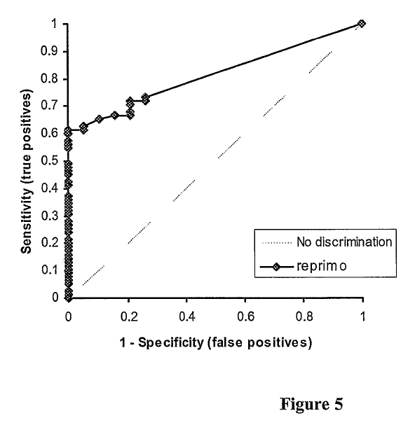

FIG. 5 shows the ROC curve analysis that was performed using the NMVs for the 75 EAC and 19 NE tissues. The area under the ROC curve (AUROC) was 0.812 (p<0.0001, 95% Confidence interval (CI); 0.73-0.90) and conveys Reprimo's accuracy in distinguishing between EAC and NE in terms of its sensitivity and specificity. The AUROC generated using the NMVs for the 75 EAC and 25 BE tissues was 0.59 (p=0.08, 95% CI; 0.47-0.71), suggesting that Reprimo methylation may indeed underscore a similarity between the two tissue types.

qMSP of Esophageal Cancer Cell Lines

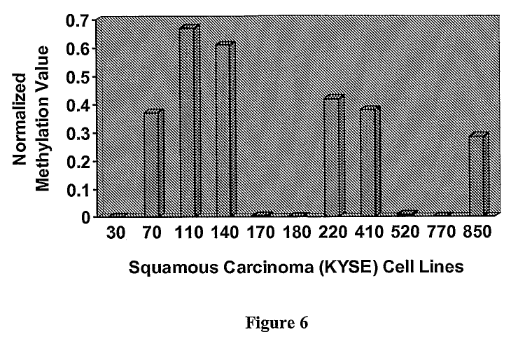

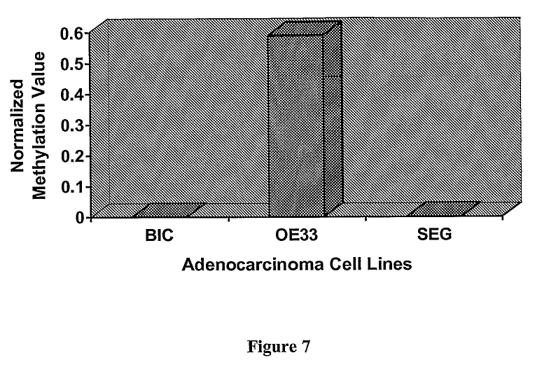

Eleven ESCC cell lines and three EAC cell lines were tested for Reprimo methylation. Seven of the 11 ESCC cell lines were methylated and 1 of the 3 EAC cell lines were methylated. The qMSP results are displayed graphically in FIGS. 6 and 7 The raw MSP data may be found in Table VIII. KYSE 110 was found to have the highest level (0.67) of Reprimo methylation of all the ESCC cell lines, while OE33 was the EAC cell line with the highest amount (0.59) of Reprimo methylation. These two cell lines were chosen for treatment with 5-Aza-2′-Deoxycytidine (5AzaC).

TABLE VIII
Quantitative MSP of Esophageal Cancer Cell Lines

| Cell Line | Tissue | qMSP Value |
|---|---|---|
| KYSE 30 | ESCC* | 0.015 |
| KYSE 70 | ESCC | 0.37 |
| KYSE 110 | ESCC | 0.67 |
| KYSE 140 | ESCC | 0.61 |
| KYSE 170 | ESCC | 0.0054 |
| KYSE 180 | ESCC | 0.0 |
| KYSE 220 | ESCC | 0.42 |
| KYSE 410 | ESCC | 0.38 |
| KYSE 520 | ESCC | 0.087 |
| KYSE 770 | ESCC | 0.001 |
| KYSE 850 | ESCC | 0.285 |
| BIC | EAC | 0.0 |
| OE33 | EAC | 0.59 |
| SEG | EAC | 0.0007 |

*ESCC: squamous cell cancer; EAC: adenocarcinoma

### EXAMPLE 3

### 5-Aza-2′-Deoxycytidine (5AzaC) Treatment of Esophageal Cancer Cell Lines

To demonstrate the gene-silencing effect of Reprimo methylation in esophageal carcinoma, two cancer cell lines were subjected to treatment with 5AzaC (Sigma, St. Louis, Mo.). The squamous carcinoma (KYSE 110) and the adenocarcinoma cell line (OE33), which demonstrated the highest quantitative values of methylation in their respective tissue types, were chosen for 5AzaC treatment. This treatment protocol has been published previously (Shibata, D. M., et al., Cancer Res., 62:5637-5640 (2002)). Briefly, 1×10⁵ cells/ml were seeded in 100-mm culture dishes and grown in a mixture of 47.5% RPMI-1640 medium (Life Technologies Inc., Rockville, Md.), 47.5% HAM's medium (Invitrogen, Carlsbad, Calif.), and 5% fetal bovine serum (Invitrogen). Cell cultures were incubated at 5% CO₂ for 24 hours at 37° C. Then, 1 μl of 5 mM 5AzaC per ml of cells was added every 24 hours for 6 days. Cells were harvested at day 0, day 2, day 4, and day 6. Harvested cells were stored at −20° C. until DNA extraction. Medium was changed every 72 hours.

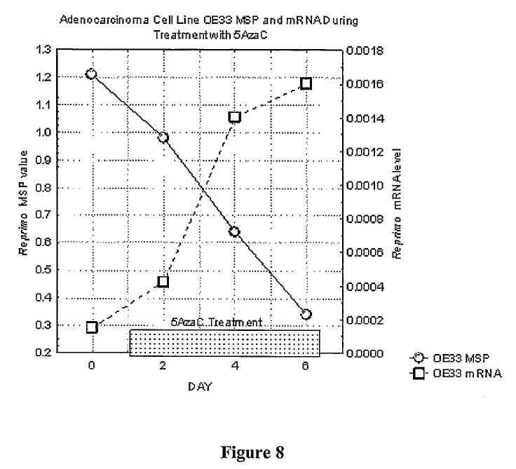

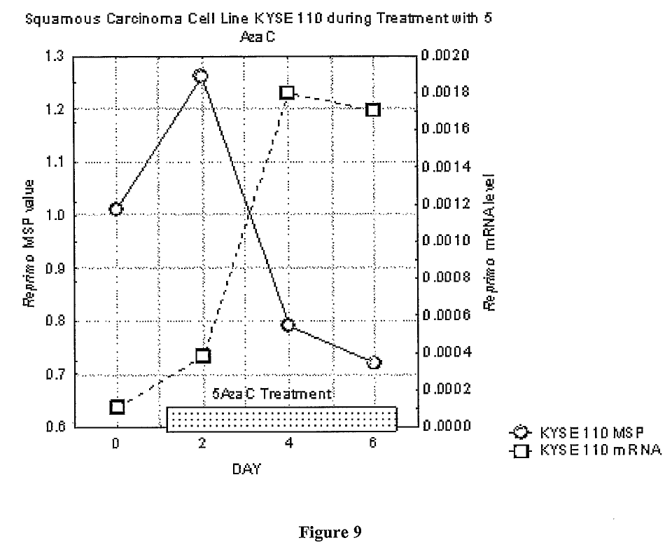

QMSP was performed on DNA from the cell lines KYSEL110 and OE33 after treatment with 5 mM 5AzaC. Real-time PCR for Reprimo RNA was performed on samples harvested at identical time points. Quantitative results for the MSP and the RT-PCR reactions are displayed in FIGS. 8 and 9. An inverse relationship between Reprimo methylation and mRNA expression was observed when the cell lines were treated with the de-methylating agent.

Statistical Analysis

The NMVs for each tissue type were compared to each other using Student's paired t-test (Statistica 6.0). In addition, to provide further statistical rigor, and because the data were asymmetrical and did not fit a normal Gaussian distribution (e.g., many datapoints were equal to zero), further non-parametric testing was performed using Mann-Whitney's U test (Statistica 6.0). To demonstrate the ability of Reprimo methylation to distinguish between NE and EAC, receiver-operator characteristic (ROC) curve analysis (25)(Analyze-it) was performed using the NMV of the 75 EAC and 19 NE tissues (See Sharma P. et al., Am J Gastroenterol. 93:1033-1036 (1998), which is hereby incorporated by reference). A p value of less than 0.05 was considered to be significant for all statistical calculations.

FIGS. 8 and 9 illustrate that methylation of Reprimo in EAC and ESCC cell lines was associated with reduced expression of Reprimo mRNA and that treatment with a demethylating agent lead to increased Reprimo mRNA expression and a concomitant reduction in Reprimo methylation. These data suggest that hypermethylation constitutes a mechanism by which Reprimo expression is silenced. In addition, 5AzaC is shown to be a potential therapeutic anti-cancer drugs (See Lemaire M. et al., Anticancer Drugs, 16:301-308 (2005) and Ahuja N., Cancer Res. 58:5489-494 (1998), incorporated by reference), and Reprimo thus represents a novel potential target for molecular-based therapies involving demethylation. The p-values of EAC vs. ESCC by Mann-Whitney testing was 0.000002, for HGD vs. ESCC 0.002, and for BE vs. ESCC 0.0001, which suggest a highly significant tendency for Reprimo methylation to target specialized columnar rather than squamous human esophageal cells in vivo.

It appears that Reprimo methylation occurs commonly in premalignant BE, in particular, long-segment BE, as well as in HGD and EAC. The level and frequency of Reprimo methylation increase in a stepwise fashion along the progression cascade toward the development of EAC. Methylation of Reprimo was not commonly detected in ESCC or in NE, suggesting this represents a cell type-specific biomarker for EAC moreso than ESCC. Further large-scale prospective longitudinal validation studies of this biomarker in progression from BE to HGD or EAC are supported by these data.

What is claimed is:

1. A method for predicting the responsiveness of a subject to a therapeutic regimen, said method comprising
a) determining a methylation status of a panel of genes in a test subject; and
b) using said test subject's methylation status of said panel of genes as indicative of said test subject's response to said therapeutic regimen.

2. The method of claim 1, wherein said therapeutic regimen comprises chemotherapy.

3. The method of claim 1, wherein said therapeutic regimen comprises radiation therapy.

4. The method of claim 3, wherein said therapeutic regimen further comprises chemotherapy.

5. The method of claim 1, wherein said subject is being treated for neoplasm.

6. The method of claim 5, wherein said neoplasm is a carcinoma.

7. The method of claim 6, wherein said carcinoma is esophageal carcinoma.

8. The method of claim 1, wherein said determining said methylation status comprises using an assay selected from the group consisting of Southern blotting, single nucleotide primer extension, methylation-specific polymerase chain reaction (MSPCR), restriction landmark genomic scanning for methylation (RLGS-M), CpG island micro array, SNUPE, and COBRA.

9. The method of claim 1, wherein said determining the methylation status of a panel of genes comprises determining the methylation status of at least one non-coding portion of at least one member of said panel of genes.

10. The method of claim 9, wherein said at least one non-coding portion comprises a gene promoter.

11. The method of claim 9, wherein said determining the methylation status of a panel of genes comprises determining the methylation status of the non-coding portions of all members of said panel genes.

12. The method claim 11, wherein said non-coding portions comprise gene promoters.

13. The method of claim 1, wherein said panel of genes comprises at least a number of genes selected from the group consisting of 3, 4, 5, 6, 7, 8, 9, 10, 11, 12, 13, 14, 15, 16, 17, 18, 19 and 20 genes.

14. The method of claim 1, wherein said panel of genes comprises at least 3 genes.

15. The method of claim 1, wherein said panel of genes comprises at least 4 genes.

16. The method of claim 1, wherein said panel of genes comprises at least 5 genes.

17. The method of claim 1, wherein said panel of genes comprises at least 6 genes.

18. The method of claim 1, wherein said panel of genes comprises at least 7 genes.

19. The method of claim 1, wherein said panel of genes comprises at least 8 genes.

20. The method of claim 1, wherein said panel of genes comprises at least 9 genes.

21. The method of claim 1, wherein said panel of genes comprises at least one gene involved in cell cycle regulation.

22. The method of claim 1, wherein said panel of genes comprises at least one gene that causes cell cycle arrest at the G1/S phase.

23. The method of claim 1, wherein said panel of genes comprises at least one gene that is responsible for a delay in chromosomal condensation during prophase.

24. The method of claim 1, wherein said panel of genes comprises at least one gene that is a regulator of p53-mediated cell cycle arrest during G2/M.

25. The method of claim 1, wherein said panel of genes comprises at least one gene that is involved in angiogenesis.

26. The method of claim 1, wherein said panel of genes comprises at least one repair gene.

27. The method of claim 1, wherein said panel of genes comprises at least one gene that encodes a receptor.

28. The method of claim 14, wherein said panel of genes comprises a combination of at least 3 genes selected from the group consisting of Reprimo, p16, TIMP-3, MGMT, Hpp-1, and CHFR.

29. The method of claim 15, wherein said panel of genes comprises a combination of at least 4 genes selected from the group consisting of Reprimo, p16, TIMP-3, MGMT, Hpp-1, and CHFR.

30. The method of claim 16, wherein said panel of genes comprises a combination of at least 5 genes selected from the group consisting of Reprimo, p16, TIMP-3, MGMT, Hpp-1, and CHFR.

31. The method of claim 17, wherein said panel of genes comprises Reprimo, p16, TIMP-3, MGMT, Hpp-1, and CHFR.

32. The method of claim 1, wherein said one or more test subjects is a population of responder subjects.

33. The method of claim 1, wherein said one or more test subjects is a responder subject.

34. The method of claim 1, further comprising the comparing the methylation status of said panel of genes to one or more test subjects and using said comparison as indicative of a test subject's responsiveness to said therapeutic regimen.

35. A method of customizing a therapeutic regimen for a subject in need thereof, said method comprising
a) determining a methylation status of a panel of genes in a test subject;
b) using said test subject's methylation status of said panel of genes as indicative of said test subject's response to said therapeutic regimen; and
c) determining an appropriate therapeutic regimen for said test subject based upon said test subject's methylation status.

36. The method of claim 35, wherein said therapeutic regimen comprises chemotherapy.

37. The method of claim 35, wherein said therapeutic regimen comprises radiation therapy.

38. The method of claim 37, wherein said therapeutic regimen further comprises chemotherapy.

39. The method of claim 35, wherein said subject is being treated for neoplasm.

40. The method of claim 39, wherein said neoplasm is a carcinoma.

41. The method of claim 40, wherein said carcinoma is esophageal carcinoma.

42. The method of claim 35, wherein said determining said methylation status comprises using an assay selected from the group consisting of Southern blotting, single nucleotide primer extension, methylation-specific polymerase chain reaction (MSPCR), restriction landmark genomic scanning for methylation (RLGS-M), CpG island micro array, SNUPE, and COBRA.

43. The method of claim 35, wherein said determining the methylation status of a panel of genes comprises determining the methylation status of at least one non-coding portion of at least one member of said panel of genes.

44. The method of claim 43, wherein said at least one non-coding portion comprises a gene promoter.

45. The method of claim 43, wherein said determining the methylation status of a panel of genes comprises determining the methylation status of the non-coding portions of all members of said panel genes.

46. The method claim 45, wherein said non-coding portions are gene promoters.

47. The method of claim 35, wherein said panel of genes comprises at least a number of genes selected from the group consisting of 3, 4, 5, 6, 7, 8, 9, 10, 11, 12, 13, 14, 15, 16, 17, 18, 19 and 20 genes.

48. The method of claim 35, wherein said panel of genes comprises at least 3 genes.

49. The method of claim 35, wherein said panel of genes comprises at least 4 genes.

50. The method of claim 35, wherein said panel of genes comprises at least 5 genes.

51. The method of claim 35, wherein said panel of genes comprises at least 6 genes.

52. The method of claim 35, wherein said panel of genes comprises at least 7 genes.

53. The method of claim 35, wherein said panel of genes comprises at least 8 genes.

54. The method of claim 35, wherein said panel of genes comprises at least 9 genes.

55. The method of claim 48, wherein said panel of genes comprises a combination of at least 3 genes selected from the group consisting of Reprimo, p16, TIMP-3, MGMT, Hpp-1, and CHFR.

56. The method of claim 49, wherein said panel of genes comprises a combination of at least 4 genes selected from the group consisting of Reprimo, p16, TIMP-3, MGMT, Hpp-1, and CHFR.

57. The method of claim 50, wherein said panel of genes comprises a combination of at least 5 genes selected from the group consisting of Reprimo, p16, TIMP-3, MGMT, Hpp-1, and CHFR.

58. The method of claim 51, wherein said panel of genes comprises Reprimo, p16, TIMP-3, MGMT, Hpp-1, and CHFR.

59. The method of claim 35, wherein said one or more test subjects is a population of responder subjects.

60. The claim 35, wherein said one or more test subjects is a responder subject.

61. A method of monitoring the progression of a disease state in a subject, said method comprising
a) determining a methylation status of a gene or a panel of genes in a test subject at a first time point;
b) determining the methylation status of said gene or said panel of genes in said test subject at a second time point; and
c) comparing the methylation status of said subject at said first and second time points to determine a difference in methylation status of said gene or said panel of genes over time;
wherein a difference in methylation status in said gene or said panel of genes over time is indicative of the progression of said disease state.

62. The method of claim 61, wherein disease state comprises neoplastic growth.

63. The method of claim 62, wherein said neoplastic growth comprises a carcinoma.

64. The method of claim 63, wherein said carcinoma is esophageal carcinoma.

65. The method of claim 61, wherein said determining said methylation status comprises using an assay selected from the group consisting of Southern blotting, single nucleotide primer extension, methylation-specific polymerase chain reaction (MSPCR), restriction landmark genomic scanning for methylation (RLGS-M), CpG island micro array, SNUPE, and COBRA.

66. The method of claim 61, wherein said determining the methylation status of a gene or a panel of genes comprises determining the methylation status of at least one non-coding portion of at least one member of said gene or said panel of genes.

67. The method of claim 66, wherein said at least one non-coding portion comprises a gene promoter.

68. The method of claim 66, wherein said determining the methylation status of a panel of genes comprises determining the methylation status of the non-coding portions of all members of said panel genes.

69. The method claim 68, wherein said non-coding portions are gene promoters.

70. The method of claim 61, wherein the methylation status is for a single gene.

71. The method of claim 71, wherein the single gene is Reprimo.

72. The method of claim 61, wherein said panel of genes comprises at least a number of genes selected from the group consisting of 3, 4, 5, 6, 7, 8, 9, 10, 11, 12, 13, 14, 15, 16, 17, 18, 19 and 20 genes.

73. The method of claim 72, wherein said panel of genes comprises a combination of at least 3 genes selected from the group consisting of Reprimo, p16, TIMP-3, MGMT, Hpp-1, and CHFR.

74. The method of claim 73, wherein said panel of genes comprises a combination of at least 4 genes selected from the group consisting of Reprimo, p16, TIMP-3, MGMT, Hpp-1, and CHFR.

75. The method of claim 74, wherein said panel of genes comprises a combination of at least 5 genes selected from the group consisting of Reprimo, p16, TIMP-3, MGMT, Hpp-1, and CHFR.

76. The method of claim 75, wherein said panel of genes comprises Reprimo, p16, TIMP-3, MGMT, Hpp-1, and CHFR.

77. The method of claim 61, wherein an increase in said methylation status over time is indicative that said disease state is not regressing.

78. The method of claim 77, wherein an increase in said methylation status over time is indicative that said disease state is progressing.

79. A method of diagnosing a disease state in a subject suspected of having a disease, said method comprising
a) determining a methylation status of a gene or panel of genes in a test subject;
b) using said test subject's methylation status of said gene panel of genes as indicative of said test subject's disease state.

80. The method of claim 79, wherein said disease state is a neoplasm.

81. The method of claim 80, wherein said neoplasm is a carcinoma.

82. The method of claim 81, wherein said carcinoma is esophageal carcinoma.

83. The method of claim 82, wherein said determining said methylation status comprises using an assay selected from the group consisting of Southern blotting, single nucleotide primer extension, methylation-specific polymerase chain reaction (MSPCR), restriction landmark genomic scanning for methylation (RLGS-M), CpG island microarray, SNUPE, COBRA.

84. The method of claim 79, wherein said determining the methylation status of a panel of genes comprises determining the methylation status of at least one non-coding portion of at least one member of said panel of genes.

85. The method of claim 84, wherein said at least one non-coding portion comprises a gene promoter.

86. The method of claim 84, wherein said determining the methylation status of a panel of genes comprises determining the methylation status of the non-coding portions of all members of said panel genes.

87. The method claim 86, wherein said non-coding portions are gene promoters.

88. The method of claim 79, wherein said panel of genes comprises at least a number of genes selected from the group consisting of 3, 4, 5, 6, 7, 8, 9, 10, 11, 12, 13, 14, 15, 16, 17, 18, 19 and 20 genes.

89. The method of claim 88, wherein said panel of genes comprises a combination of at least 3 genes selected from the group consisting of Reprimo, p16, TIMP-3, MGMT, Hpp-1, and CHFR.

90. The method of claim 89, wherein said panel of genes comprises a combination of at least 4 genes selected from the group consisting of Reprimo, p16, TIMP-3, MGMT, Hpp-1, and CHFR.

91. The method of claim 90, wherein said panel of genes comprises a combination of at least 5 genes selected from the group consisting of Reprimo, p16, TIMP-3, MGMT, Hpp-1, and CHFR.

92. The method of claim 91, wherein said panel of genes comprises Reprimo, p16, TIMP-3, MGMT, Hpp-1, and CHFR.

93. The method of claim 92, wherein said one or more test subjects is a population of test subjects.

94. The claim 79, wherein said one or more test subjects is a single test subject.

95. A method of predicting the recurrence of a disease state in a subject confirmed with previously having said disease, said method comprising:
a) determining a methylation status of a panel of genes in a test subject; and
b) using said test subject's methylation status of said panel of genes as indicative of said test subject's probability of said disease state reoccurring.

96. The method of claim 95, wherein said subject said disease state is neoplasm.

97. The method of claim 97, wherein said neoplasm is a carcinoma.

98. The method of claim 97, wherein said carcinoma is esophageal carcinoma.

99. The method of claim 98, wherein said determining said methylation status comprises using an assay selected from the group consisting of Southern blotting, single nucleotide primer extension, methylation-specific polymerase chain reaction (MSPCR), restriction landmark genomic scanning for methylation (RLGS-M), CpG island microarray, SNUPE, and COBRA.

100. The method of claim 99, wherein said determining the methylation status of a panel of genes comprises determining the methylation status of at least one non-coding portion of at least one member of said panel of genes.

101. The method of claim 100, wherein said non-coding portion is a gene promoter.

102. The method of claim 100, wherein said determining the methylation status of a panel of genes comprises determining the methylation status of the non-coding portions of all members of said panel genes.

103. The method claim 102, wherein said non-coding portions are gene promoters.

104. The method of claim 102, wherein said panel of genes comprises at least a number of genes selected from the group consisting of 3, 4, 5, 6, 7, 8, 9, 10, 11, 12, 13, 14, 15, 16, 17, 18, 19 and 20 genes.

105. The method of claim 104, wherein said panel of genes comprises a combination of at least 3 genes selected from the group consisting of Reprimo, p16, TIMP-3, MGMT, Hpp-1, and CHFR.

106. The method of claim 105, wherein said panel of genes comprises a combination of at least 4 genes selected from the group consisting of Reprimo, p16, TIMP-3, MGMT, Hpp-1, and CHFR.

107. The method of claim 106, wherein said panel of genes comprises a combination of at least 5 genes selected from the group consisting of Reprimo, p16, TIMP-3, MGMT, Hpp-1, and CHFR.

108. The method of claim 107, wherein said panel of genes comprises Reprimo, p16, TIMP-3, MGMT, Hpp-1, and CHFR.
